(19) United States
(12) Patent Application Publication
(10) Pub. No.: US 2011/0082389 A1
(43) Pub. Date: Apr. 7, 2011

(54) APPARATUS AND METHOD FOR ASSESSING CONDITION OF ARTICULAR CARTILAGE

(75) Inventors: WILLIAM JOSEPH McCARTY, San Diego, CA (US); PRIYA SUNDARAMURTHY, Fremont, CA (US); CARYN URBANCZYK, San Diego, CA (US); ATAL PATEL, Mission Viejo, CA (US); JACOB HAHR, Trabuco Canyon, CA (US); ROBERT L. SAH, San Diego, CA (US); MOHAMMAD SOTOUDEH, San Diego, CA (US); ANTHONY RATCLIFFE, San Diego, CA (US)

(73) Assignees: THE REGENTS OF THE UNIVERSITY OF CALIFORNIA, Oakland, CA (US); SYNTHASOME, INC., San Diego, CA (US)

(21) Appl. No.: 12/899,031

(22) Filed: Oct. 6, 2010

Related U.S. Application Data
(60) Provisional application No. 61/249,339, filed on Oct. 7, 2009.

(51) Int. Cl. A61B 5/00

(52) U.S. Cl. 600/578

(57) ABSTRACT

A measuring system may provide quantitative information relating to condition of cartilage. Negative pressure may be applied to cartilage to induce flow of fluid from or through the cartilage. A level of negative pressure needed to induce a particular flow of the fluid may be employed to provide a quantitative indicia of cartilage condition. An averaged level of negative pressure measured over a period of time may be used to calculate hydraulic resistance of the cartilage.

### RELATED APPLICATIONS

This application claims the priority date of U.S. Provisional Application No. 61/249,339 filed Oct. 7, 2009.

### STATEMENT REGARDING FEDERALLY SPONSORED RESEARCH OR DEVELOPMENT

This invention was made with Government support under AR044058 awarded by NIH. The Government has certain rights in the invention.

### BACKGROUND OF THE INVENTION

The present invention relates to diagnosis and repair of defects and damage of cartilage tissue.

Articular cartilage is a load-bearing connective tissue at the ends of long bones in synovial joints that facilitates low-friction, low-wear joint articulation. The load-bearing ability of cartilage is dependent on the presence of a large aggregating proteoglycan, aggrecan, in a matrix structure. Aggrecan is highly negatively charged due to its numerous glycosaminoglycan (GAG) side chains, and the charge density of these GAG molecules creates a swelling pressure in the interstitial fluid of cartilage that resists compression.

It is known that hydraulic permeability of osteochondral tissue, especially articular cartilage, may increase with degeneration and erosion. Progressive degeneration and erosion of articular cartilage that can occur with osteoarthritis (OA) has been correlated with increased hydraulic permeability. In addition, focal defects, which are commonly observed in the knees of symptomatic patients during arthroscopy, are discrete areas of cartilage erosion that also likely have increased hydraulic permeability. Increase in hydraulic permeability may diminish the ability of cartilage to maintain fluid pressurization, leading to larger strains on the cartilage matrix and further degeneration, as well as abnormal fluid flow and communication between the intraarticular space and the subchondral bone.

Repair strategies for cartilage defects may include arthroscopic procedures, such as microfracture; soft tissue grafts; osteochondral grafts of autogenic or allogenic source material; cell transplantation with or without a scaffold, including autologous cell implantation and mesenchymal stem cells; and synthetic and natural scaffolds. Interstitial fluid pressurization, and load-bearing capacity, may be typically restored with osteochondral graft techniques.

Determination of the possible presence and extent of cartilage defects is a critical factor in formulating a repair strategy. Clinically useful measures to diagnose the extent of cartilage degeneration and efficacy of repair strategies are limited, especially with regard to pressure maintenance within the cartilage tissue. Presently used techniques may include visual observation during arthroscopy and/or imaging modalities such as plain film x-ray attenuation, magnetic resonance imaging (MRI) and computed tomography (CT). These methods alone may produce only limited quantitative results.

While a determination of hydraulic permeability may be a valuable indicator of condition of cartilage, a direct measurement of this parameter has been performed only by experimental perfusion techniques on isolated samples of tissue in a laboratory setting. A typical ex vivo permeability measurement must be performed for a number of hours before yielding meaningful results. Indirect measurements of hydraulic permeability, extrapolated from mechanical indentation testers, have been performed, but require tissue deformation and assumptions about matrix composition that may not be applicable in damaged tissues. Direct measurement of hydraulic permeability, in situ, has heretofore not been practicable.

As can be seen, there is a need for a system that may produce quantitative information indicative of cartilage condition. In particular there is a need for a system in which a quantitative indicator of hydraulic permeability may be determined in situ.

### SUMMARY OF THE INVENTION

In one aspect of the present invention, a system for assessing condition of cartilage may comprise: a contact device; a flow inducer; a fluid circuit interconnecting the contact device and the flow inducer; and a pressure sensor for determining negative pressure (−P) in the fluid circuit.

In another aspect of the present invention, a contact device for a system for assessing condition of cartilage may comprise: a flexible cap; and a flexible tube attached to the cap through which negative pressure can be applied to the cartilage when the cap is in contact with the cartilage.

In still another aspect of the present invention, a method for evaluating condition of cartilage may comprise the steps of: inducing flow of fluid from or through the cartilage; measuring negative pressure required to produce a particular flow rate of the fluid; and employing the measured negative pressure to produce a quantitative assessment of condition of the cartilage.

These and other features, aspects and advantages of the present invention will become better understood with reference to the following drawings, description and claims.

### BRIEF DESCRIPTION OF THE DRAWINGS

FIG. 1 is a schematic diagram of system for assessing condition of cartilage in accordance with an embodiment of the invention;

FIG. 2 is set of graph curves that illustrate, comparatively, data from healthy and defective cartilage in accordance with an embodiment of the invention;

FIG. 3 is set of bar graphs that illustrate, comparatively, hydraulic resistance of healthy and defective cartilage in accordance with an embodiment of the invention;

FIG. 4 is a perspective top view of a contact device in accordance with the invention;

FIG. 5 is a perspective bottom view of the contact device of FIG. 4 in accordance with the invention; and

FIG. 6 is a flow chart of a method for assessing condition of cartilage in accordance with an embodiment of the invention.

### DETAILED DESCRIPTION OF THE INVENTION

The following detailed description is of the best currently contemplated modes of carrying out the invention. The description is not to be taken in a limiting sense, but is made merely for the purpose of illustrating the general principles of the invention, since the scope of the invention is best defined by the appended claims.

Various inventive features are described below that can each be used independently of one another or in combination with other features.

The present invention generally provides a system that allows for in situ determination of hydraulic resistance to assess if a defect or damage to cartilage is present. In a particular application, the measurement system may be used in an arthroscopic setting.

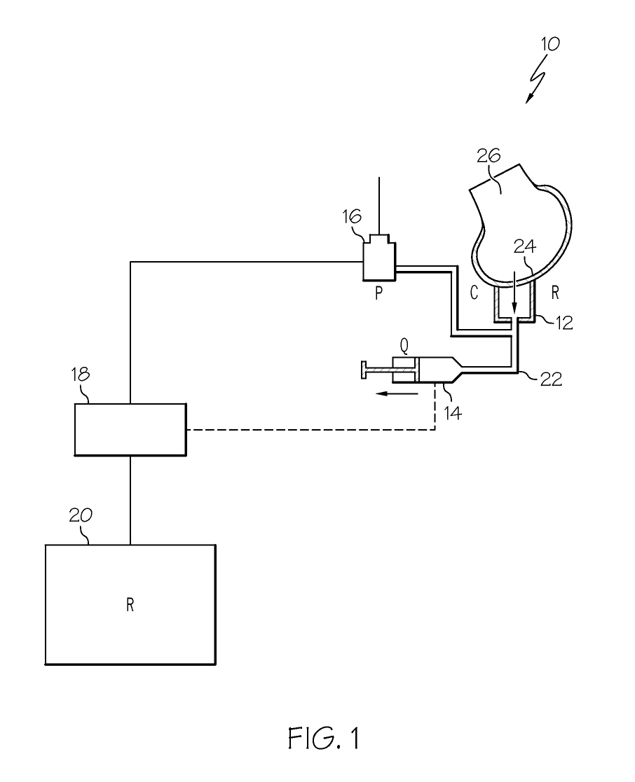

Referring now to FIG. 1, an exemplary embodiment of the present invention is shown. A measurement system, designated generally by the numeral 10, may comprise a contact device 12, a flow inducer 14, a pressure sensor 16, a processor 18 and a display unit 20. The contact device 12, the flow inducer 14 and the pressure sensor 16 may be interconnected with each other on a fluid circuit 22. In an exemplary embodiment of the system 10, the processor 18 may be electrically connected to the pressure sensor 16. In another embodiment of the system 10, the processor 18 may be electrically connected to both the pressure sensor 16 and the flow inducer 14.

In operation, the contact device 12 is in contact with a region of osteochondral tissue 24 such as cartilage, which may be referred to hereinafter as cartilage 24. The flow inducer 14 may be employed to produce negative pressure in the fluid circuit 22. The contact device 12 may apply this negative pressure to the cartilage 24. In response to the negative pressure, fluid 26 may flow through or out of the cartilage. The fluid 26 may comprise any one of various fluids (e.g., cartilage interstitial fluid, synovial fluid, and/or the fluid component of bone marrow) which may be present in or adjacent to the cartilage 24.

As the fluid 26 may flow out of or through the cartilage 24, it may increase overall volume of fluid in the fluid circuit 22. A rate at which this volume increases (Q) may be a function of magnitude of negative pressure (−P) in the fluid circuit 22. In other words a flow rate (i.e., Q) of the fluid 26 may be interrelated to −P. It must be noted, however, that the rate Q is not exclusively determined by the pressure level P. One factor that affects rate Q is cartilage hydraulic pressure. If the cartilage 24 has a high hydraulic resistance (i.e., low permeability), then a particular rate of flow Q may require a high magnitude of −P. Conversely, if the cartilage 24 has a low hydraulic resistance (i.e., high permeability), then the same Q may be attainable with a lower magnitude of −P. It may be seen that, by operation of the system 10, a value of hydraulic resistance (R) of a particular portion of the cartilage 24 may be quantified with a determination of two measurable parameters, −P and Q.

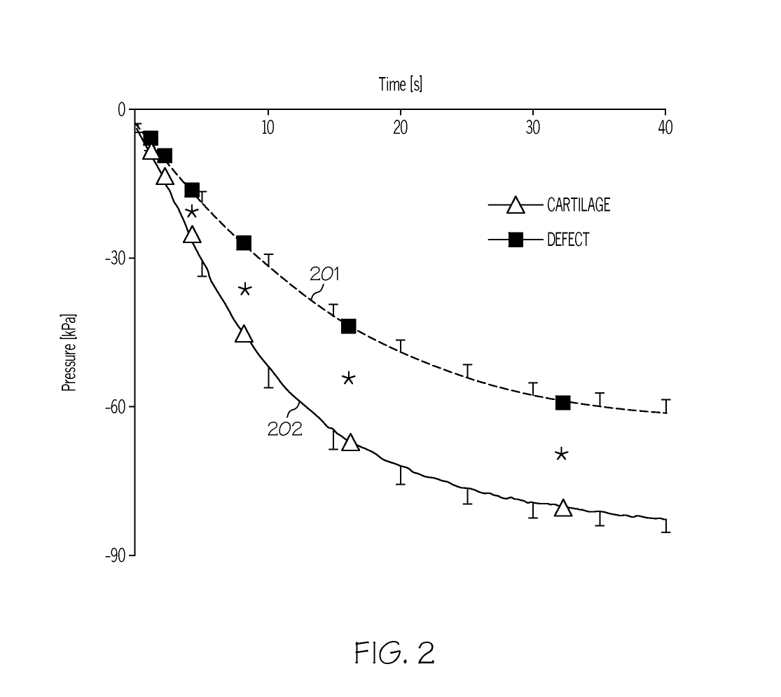

Referring now to FIG. 2, a series of graph curves were prepared to illustrate comparatively how the system 10 responded while being applied to healthy cartilage and defective cartilage. A curve 202 was prepared to illustrate how −P varied over time when the contact device 12 was applied to a healthy portion of the cartilage 24. A curve 201 was prepared to illustrate how −P may varied over time when the contact device 12 was applied to a defective portion of the cartilage 24. Both of the curves 201 and 202 represent operation of the system 10 when Q was held constant and equal for both curves. It may be seen that with progressive time lapse, the curve 202 (i.e., healthy cartilage) shows a higher magnitude of negative pressure than the curve 201 (i.e., defective cartilage). It may also be seen that with increasing time, a pressure difference (ΔP) between curves 201 and 201 became larger. For example, at a time of about 4 seconds ΔP was about 2 kilopascals (kPa) to about 3 kPa. At a time of about 20 seconds ΔP was about 30 kPa. Thus at 20 seconds, ΔP was more readily discernible than at 4 seconds.

It has been found that data relating to −P may be reduced and fit to a model equation to determine R of cartilage 24 in various states of health or degeneration. Pressure values may be normalized to a baseline average pressure recorded over 5 seconds before initiation of flow. A zero time point may be set as a point at which the pressure may increase over standard deviations from the baseline average. The system 10 may be modeled in accordance with the expression:

$$- P = QR ( 1 - exp [ - \frac{t}{RC} ] ) \qquad Equation 1$$

where:

−P is averaged measured pressure over a time t;

R is hydraulic resistance of the cartilage;

C is a predetermined compliance of the system 10; and

Q is the flow rate.

In a typical measurement of cartilage condition, the time t may be selected to be consistent with a time constant of the system 10 in its operational mode. In other words, the time t may be selected to be equal to R*C. In such a case, the exponential term of Equation 1 may be exp (−1) or about 0.36. This may correspond to a time t at which −P may be at about 64% of its final value. When the time t is selected to be equal to the time constant, the system 10 may be operated in a manner that may clinically optimized. In other words, values of −P may be readily discernible for comparative purposes but time lapse for performing a measurement may remain desirably low. For example, it has been found that an optimum time for performing a particular measurement sequence with an exemplary embodiment of the system 10 may be about 20 seconds.

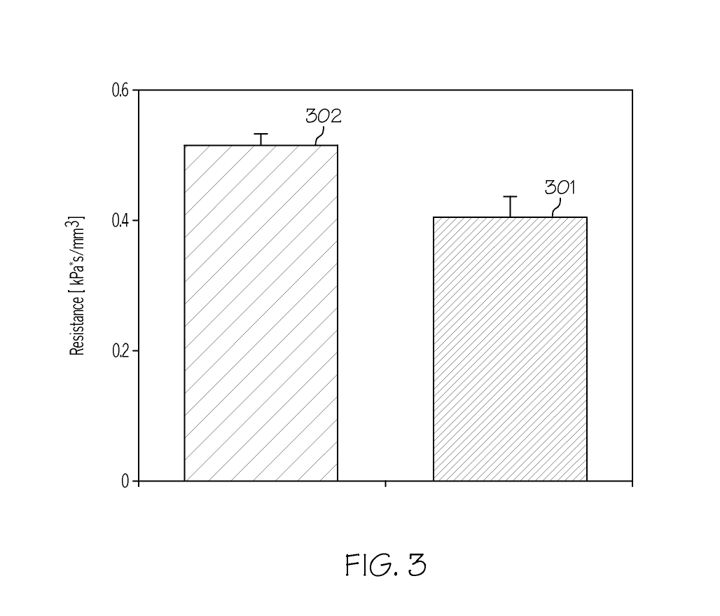

Referring now to FIG. 3, a bar graph shows a comparative set of R values that were determined from the data of FIG. 2. A bar graph 301 represents a value of R that was determined by applying the contact device 12 to defective cartilage. A bar graph 302 represents a value of R that was determined by applying the contact device 12 to healthy cartilage.

In operation, the contact device 12 may be applied to cartilage in any one of numerous clinical settings, such as open-joint surgery or arthroscopic surgery. The system 10 may be employed during a procedure to determine, in “real time” whether a patient's cartilage is healthy or defective. The contact device may be applied to a particular portion of the patient's cartilage and, within about 20 seconds, a clinician may be able to see a quantitative display of R on the display unit 20. In this context, the system 10 may be employed as an adjunct to a cartilage repair procedure. Because cartilage condition may be determined in “real-time”, repair strategy decisions may be made based on quantitative assessment of cartilage conditions.

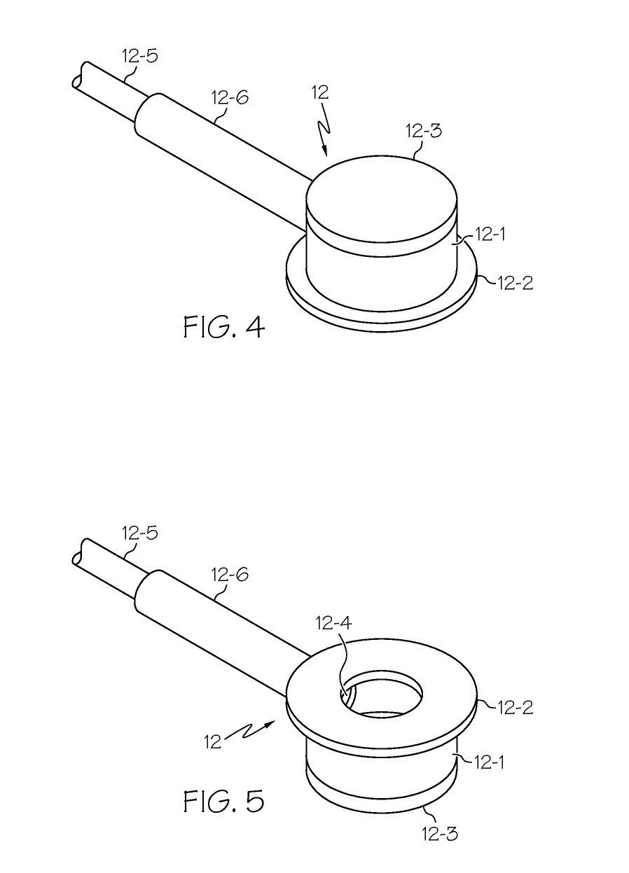

Referring now to FIGS. 4 and 5, an exemplary embodiment of the contact device 12 is illustrated. In the exemplary embodiment of FIGS. 4 and 5 the contact device 12 may be constructed so that it may be arthroscopically-deliverable. The contact device 12 may comprise a cylindrical silicone rubber cap 12-1, having exemplary dimensions of about 8 millimeters (mm) height by 15 mm inner diameter by 1.4 mm thickness. The cap 12-1 may be glued on a flat silicone toroidal ring 12-2 having exemplary dimensions of about 18 mm outer diameter, 10 mm inner diameter by 1.0 mm thickness. A silicone rubber disc 12-3 having exemplary dimensions of about 23 mm diameter by 2.0 mm thick may be glued to the closed end. A hole 12-4, having an exemplary dimension of about 3 mm diameter, may be in a side of the cap 12-1. A stainless steel tube 12-5 (e.g., 4.5 mm outer diameter by 0.13 mm thick) may be adhered inside silicone tubing 12-6 (e.g., 3.8 mm inner diameter by 1.0 mm thick) and secured to the hole 12-4. The contact device 12, constructed with dimensions and materials described above, may be suitable for insertion through a standard arthroscopic cannula with an inner diameter of about 8 mm. While the contact device 12 may be compressed to pass through the cannula, the device 12 may be rigid enough to resist deformation when exposed to negative pressure, for example as high as 80 kPa, when in contact with the cartilage 24.

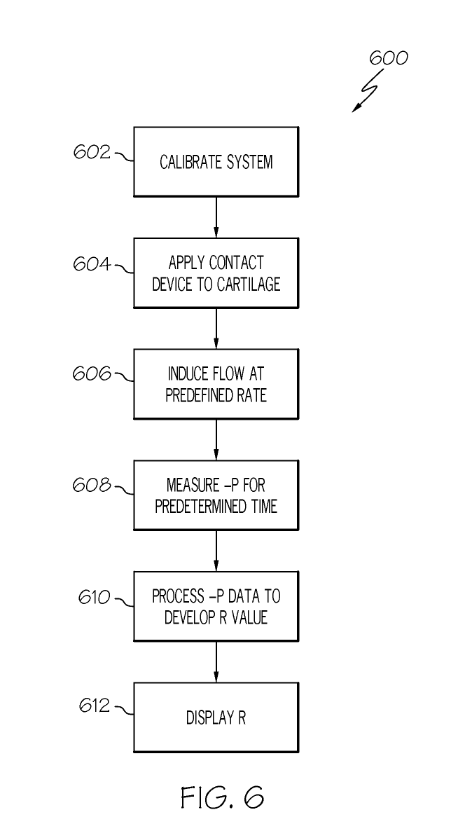

Referring now to FIG. 6, a flow chart may illustrate an exemplary method 600 which may be employed to evaluate condition of cartilage. In a step 602 a measurement system may be calibrated for a particular clinical session wherein a particular length and volume of the fluid circuit may be employed. (For example, calibration may be performed to quantify compliance (C) of the system 10. The fluid circuit 22 may be filled with fluid such as saline. The contact device 12 may be placed on a rigid surface and a compliance value, in units kPa/mm³, may be determined. The processor 18 may be programmed to apply the determined C to all R determinations for the particular clinical session).

In a step 604, the contact device may be placed in contact with a portion of cartilage of a patient (e.g., the contact device 12 may be inserted through an arthroscopic cannula and into contact with the cartilage 24 during an arthroscopic diagnostic and repair procedure). In a step 606, flow may be induced in the fluid circuit (e.g., the flow inducer 14 may be operated to induce a volume change of the system 10 at a rate Q; the rate Q may be predetermined and constant. Alternatively, the rate Q may be variable in which case the variable rate Q may be continuously transmitted to the processor 18). In a step 608, negative pressure in the system may be measured for a time t (e.g., the pressure sensor 16 may sense −P over a time period that corresponds to a time constant of the system 10). In a step 610, −P data may be processed to produce an R value (e.g., average −P collected over the time t may be processed in accordance with the Equation 1 in the processor 18 to yield an R value for a particular portion of the cartilage 24). In step 612, the R value may be displayed (e.g., the processor 18 may produce a signal to the display unit 20 so that the display unit 20 may display the R value to a clinician).

It should be understood, of course, that the foregoing relates to exemplary embodiments of the invention and that modifications may be made without departing from the spirit and scope of the invention as set forth in the following claims.

We claim:

1. A system for assessing condition of cartilage comprising:
a contact device;
a flow inducer;
a fluid circuit interconnecting the contact device and the flow inducer; and
a pressure sensor for determining negative pressure (−P) in the fluid circuit.

2. The system of claim 1 further comprising a processor for calculating a value of hydraulic resistance (R) based on the determined negative pressure.

3. The system of claim 2:
wherein the flow inducer produces a flow rate (Q) for a time period t;
wherein the −P is continuously recorded for the time period t; and
wherein an averaged value of −P for the time period t is used to calculate R.

4. The system of claim 3 wherein the processor determines R in accordance with the expression:
$$- P = QR ( 1 - exp [ - \frac{t}{RC} ] )$$
where:
−P is averaged measured pressure over the time period t;
R is hydraulic resistance of the cartilage;
C is a predetermined compliance of the system; and
Q is the flow rate.

5. The system of claim 3 wherein the time period t corresponds to a time constant of the system.

6. The system of claim 3 wherein the time period t is no greater than about 20 seconds.

7. The system of claim 1 wherein the contact device can be passed through a standard arthroscopic cannula.

8. The system of claim 1 further comprising a display unit for graphically displaying quantitative indicia of condition of the cartilage.

9. The system of claim 8 wherein the display unit displays a value of hydraulic resistance of the cartilage (R).

10. A contact device for a system for assessing condition of cartilage, comprising:
a flexible cap; and
a flexible tube attached to the cap through which negative pressure can be applied to the cartilage when the cap is in contact with the cartilage.

11. The contact device of claim 10 wherein the cap is deformable into a shape that can pass through a standard arthroscopic cannula.

12. The contact device of claim 10 wherein the cap is rigid enough to withstand negative pressure of up to 80 kilopascals (kPa) without deforming.

13. The contact device of claim 10 further comprising a flexible toroidal ring attached to and extending outwardly from the cap for sealing the cap to the cartilage.

14. A method for evaluating condition of cartilage comprising the steps of:
inducing flow of fluid from or through the cartilage;
measuring negative pressure required to produce a particular flow rate of the fluid; and
employing the measured negative pressure to produce a quantitative assessment of condition of the cartilage.

15. The method of claim 14 further comprising the step of:
placing a contact device into contact with the cartilage; and
wherein the step of producing flow comprises generating negative pressure in a fluid circuit that is connected to the contact device.

16. The method of claim 15 wherein the step of placing the contact device into contact with the cartilage is performed arthroscopically.

17. The method of claim 14 further comprising the step of:
averaging values of negative pressure measured over a time t; and
employing an average of the values to calculate hydraulic resistance (R) of the cartilage.

18. The method of claim 17 wherein R is determined in accordance with the expression:
$$- P = QR ( 1 - exp [ - \frac{t}{RC} ] )$$
where:
−P is averaged measured negative pressure over the time t;
R is hydraulic resistance of the cartilage;
C is a predetermined compliance of a measuring system;
and
Q is a rate of flow of the fluid.

19. The method of claim 18 wherein the time t is equivalent to a time constant of the system.

20. The method of claim 14 wherein the step of performing measurement of negative pressure is performed for no more than 20 seconds.
